(19) United States
(12) Patent Application Publication
(10) Pub. No.: US 2022/0341904 A1
(43) Pub. Date: Oct. 27, 2022

(54) DETERMINING CONCENTRATIONS OF POLYHALOGENATED COMPOUNDS

(71) Applicant: BABCOCK & WILCOX VOLUND AB, Göteborg (SE)

(72) Inventor: Sven ANDERSSON, Hisings Backa (SE)

(21) Appl. No.: 17/764,876

(22) PCT Filed: Nov. 4, 2020
(86) PCT No.: PCT/EP2020/080967

§ 371 Date: Mar. 29, 2022

(30) Foreign Application Priority Data
Nov. 8, 2019 (SE) 1951290-4

(51) Int. Cl. G01N 33/00; G01N 1/22

(52) U.S. Cl. CPC G01N 33/0049; G01N 1/2214; G01N 1/2247

(57) ABSTRACT

Method for determining a concentration of one or more polyhalogenated compounds in a gas. The method comprises the steps of exposing at least one sampler (10) containing or constituted by a material (14) comprising a polymer matrix that is suitable for absorbing one or more polyhalogenated compounds, and a filler that is suitable for absorbing and adsorbing one or more polyhalogenated compounds which is distributed through said polymer matrix, to gas (12) containing one or more polyhalogenated compounds during a sampling period, whereby said gas (12) constitutes at least part of said gas whose concentration of one or more polyhalogenated compounds is to be determined, determining an amount of one or more polyhalogenated compounds adsorbed or absorbed by said material, (14), and calculating a concentration of one or more polyhalogenated compounds in said gas (12) to which said material (14) was exposed, either upstream or downstream of said at least one sampler (10).

### TECHNICAL FIELD

The present invention concerns a method for determining a concentration of one or more polyhalogenated compounds in a gas. The present invention also concerns a computer program product comprising a computer program containing computer program code means arranged to cause a computer or a processor to execute all of the calculation steps of such a method. The present invention further concerns a new use of a known material.

### BACKGROUND OF THE INVENTION

The Stockholm Convention on Persistent Organic Pollutants (POPs) is an international environmental treaty that was signed in 2001 which aims to eliminate or restrict the production and use of POPs to protect human health, wildlife and the environment (UNEP Stockholm Convention, United Nations, 2001). Many of the POPs listed in this international environmental treaty are polyhalogenated compounds. One group of polyhalogenated compounds of particular concern is polychlorinated dibenzo-p-dioxins and dibenzofurans (PCDD/F), commonly referred to as dioxins, which are considered to be carcinogenic, extremely toxic and persistent.

U.S. Pat. No. 7,022,162 B2 discloses a material for retaining polyhalogenated compounds. The material comprises a polyolefin matrix and a filler, such as carbon, which is suitable for adsorbing one or more polyhalogenated compounds and which is uniformly incorporated and distributed through the polyolefin matrix. Tower packings, mist eliminators and dry or semi-wet fixed bed packings can be produced from this material, so that for example waste gas from an incineration process may be passed through the material and cleaned immediately after the incineration in order to reduce the gas phase concentration of one or more polyhalogenated compounds in the waste gas. Extensive experience from a large number of such installations have proven high and consistent removal efficiencies regardless of inlet concentration if the dust concentration is low, i.e. below 10 mg/Nm³ d·g., which shows that the polyhalogenated compounds mainly exist in the gas phase.

The European Standard EN1948-1:2006 entitled “Stationary source emissions—Determination of the mass concentration of PCDDs/PCDFs and dioxin-like PCBs—Part 1: Sampling of PCDDs/PCDFs” describes how sampling of PCDD/F from a gas flow can be carried out in order to determine the concentration of PCDDs/PCDFs and dioxin-like PCBs in the gas flow. This European Standard has been developed to measure PCDD/PCDF concentrations of about 0.1 ng I-TEQ/m³ (International Toxic Equivalent) in stationary source emissions. It specifies both method validation and a framework of quality control requirements which shall be fulfilled by any PCDD/PCDF sampling, although it does not indicate that one particular method should be used to measure PCDD/PCDF concentrations. A user has the possibility to choose between three different methods: a “filter/condenser method”, a “dilution method” and a “cooled probe method”. All three of these methods require that a small portion of the gas being analyzed is extracted from the main flow isokinetically, i.e. at the same gas velocity as the main flow, using a probe. The sampling of PCDDs/PCDFs and dioxin-like PCBs in accordance with European Standard EN1948-1:2006 is associated with substantial cost and effort.

The Industrial Emissions Directive 2010/75/EU of the European Parliament and of the Council of 24 Nov. 2010 on Industrial Emissions (Integrated Pollution Prevention and Control), which is currently in force, prescribes a six to eight hour PCDD/F sampling that is to be performed twice annually for Waste-to-Energy plants, such as municipal solid waste or hazardous waste incineration plants, in the European Union.

It has however been shown that PCDD/F concentrations may be several orders of magnitude larger during the initial start-up phase or after operational disturbances of a Waste-to-Energy plant (see the article by Gass et al entitled “PCDD/F-Emissions During Cold Start-up and Shut Down of a Municipal Waste Incinerator” published in Organohalogen Compounds, Vol. 56, pp. 193196 in 2002 and the article entitled “Adiox® for dioxin removal in wet scrubbers and semi-wet or dry absorbers”, Transactions on Ecology and the Environment, Vol. 109, 2008 WIT Press, Waste Management and the Environment IV 569).

Such extraordinary concentrations during a start-up phase or after operational disturbances may contribute significantly to the plant's yearly emissions to air, but sampling is normally not performed during the start-up phase and these peaks are therefore not monitored. This has led to the development of sophisticated continuous long-term sampling devices which typically use a sampling period of one month. These devices are however, associated with even more effort and cost than short term measurements conducted in accordance with European Standard EN1948-1:2006. These devices use active samplers into which a very small fraction of the entire gas is sampled: typically in the order of 1/100000th. Samples obtained from such devices are sent to a laboratory for analysis in accordance with European Standard EN1948-2:2006 and EN1948-3:2006, which can take about ten days. Such a measurement procedure does not allow an operator to control a process in real time with a view to reducing emissions of PCDD/F while sampling is taking place. Instead, an operator can only determine whether legal emission targets were met after receiving the results from the laboratory. Furthermore, since very low levels of PCDD/Fs need to be detected, laboratory results can often be inaccurate due to measurement uncertainties, and measurement errors increase as the amount of PCDD/Fs decreases.

The Waste Incineration Best Available Techniques (WI BREF/BAT) Reference Document, published by the Joint Research Centre, European IPPC Bureau, Final Draft, December 2018, states new ranges of BAT-associated environmental performance levels (BAT-AELs) and that PCDD/Fs and WHO-PCBs should be measured 1) Once every six months for short-term sampling and 2) Once every month for long-term sampling. Furthermore, “The monitoring does not apply if the emission levels are proven to be sufficiently stable.” and “The monitoring does not apply where the emissions of dioxin-like PCBs are proven to be less than 0.01 ng WHO-TEQ/Nm³.” The WI BREF also states (on page 475) that “No European Standard is available for long-term sampling”, although the proposed EN1948-5 standard for long-term sampling of PCDD/Fs and dioxin-like PCBs exists as a draft. There exists a number of commercial long-term samplers, but these are expensive to own and operate.

In light of the above, there is a need to provide a reliable and cost-effective long-term sampling method to determine the concentration of polyhalogenated compounds, such as PCDD/Fs, in a gas so that an operator can determine whether emissions are below the legal emission limit and sufficiently stable.

### SUMMARY OF THE INVENTION

An object of the invention is to provide an improved method for determining the concentration of one or more polyhalogenated compounds in a gas.

This object is achieved by a method according to claim 1. The method comprises the steps of exposing at least one sampler containing or constituted by a material comprising a polymer matrix that is suitable for absorbing one or more polyhalogenated compounds and a filler that is suitable for adsorbing one or more polyhalogenated compounds which is distributed through the polymer matrix, to a gas containing one or more polyhalogenated compounds during a sampling period (i.e. exposure time), whereby the gas to which the sampler material is exposed, i.e. the gas that comes into contact with the material of the one or more sampler, constitutes at least part of the gas whose concentration of one or more polyhalogenated compounds is to be determined, i.e. the at least one sampler's material is either exposed to all, or a substantial amount, of the gas whose concentration of one or more polyhalogenated compounds is to be determined or it is exposed to only some of that gas, namely a fraction of the total amount of gas whose concentration of one or more polyhalogenated compounds is to be determined. The method comprises the step of determining an amount of one or more polyhalogenated compounds absorbed and adsorbed by the material, by conducting a chemical analysis of the material, using Gas chromatography-mass spectrometry (GC-MS) for example, after the sampling period for example, and calculating a concentration of one or more polyhalogenated compounds in the gas to which the material was exposed, either upstream or downstream of the at least one sampler, or both upstream and downstream of the at least one sampler.

The proposed method may thus be performed in-situ, without the need to extract the gas to an external sampling device. Consequently, the method does not depend on any moving parts, such as pumps, for extracting the gas, and is therefore more robust and reliable than so-called extractive methods in which a small portion of the gas is diverted into an apparatus for analyzing the gas.

Polymers can absorb large amounts of one or more polyhalogenated compounds from gases, which can later be desorbed. In order to avoid the release of polyhalogenated compounds from the polymer, filler particles, such as carbon particles, are dispersed in a polymer matrix. Polyhalogenated compounds are firstly absorbed in the polymer matrix and they then diffuse to the surface of the filler particles, where they are practically irreversibly adsorbed. The polymer matrix acts as a selective barrier, which protects the filler from other contaminants such as Mercury. Glass fibres may also be added to the material in order to improve its mechanical properties at higher temperatures. Several types of components, such as tower packings and demisters (droplet separators) and dry fixed bed fillings can be produced from the material to make one or more samplers. The gas to which the material is exposed can either flow past or through the material of the at least one sampler. The material is preferably arranged in such a way that a large surface area of material is exposed during sampling in order to maximize the material's absorption and adsorption rate. The lifetime of the material depends on the application, but is normally 4-8 years. The lifetime of the material is limited by aging of the polymer matrix, surface scaling or polyhalogenated compound absorption and adsorption rate. Aging of the polymer matrix, which leads to embrittlement of the material, is usually the lifetime limiting factor. It is possible to incinerate the material after usage. Practically no residues are produced during the incineration of the material and the polyhalogenated compounds contained in the material are destroyed and thereby removed from the eco cycle.

If the material of one or more samplers is exposed to only some of the gas whose concentration of one or more polyhalogenated compounds is to be determined, the amount of one or more polyhalogenated compounds absorbed and adsorbed by the material can be correlated to the concentration of one or more polyhalogenated compounds in the gas whose concentration of one or more polyhalogenated compounds is to be determined. The concentration increase per time of one or more polyhalogenated compounds in the sampler material is proportional to a Time Weighted Average (TWA) concentration in the gas whose concentration of one or more polyhalogenated compounds is to be determined, which will enable an operator to then calculate or estimate the concentration of one or more polyhalogenated compounds in the gas whose concentration of one or more polyhalogenated compounds is to be determined.

If the material of one or more samplers is exposed to all, or a substantial amount of the gas whose concentration of one or more polyhalogenated compounds is to be determined, the method may comprise the steps of measuring or calculating the material's polyhalogenated compound absorbing and adsorbing efficiency, and calculating a concentration of one or more polyhalogenated compounds in the gas whose concentration of one or more polyhalogenated compounds is to be determined. A substantial amount of the gas may be at least 70%, or at least 80% or at least 90%, or at least 95% of the gas.

The material's polyhalogenated compound absorbing and adsorbing efficiency may be calculated by application of mass balance for example (i.e. by applying the law of conservation of mass to the system to account for all matter accumulating in, as well as entering and leaving the system), or by measuring the concentration of one or more polyhalogenated compounds in gas upstream of the material and in gas downstream of the material.

The method can be used to determine the PCDD/F concentration in a gas. The gas whose concentration of one or more polyhalogenated compounds is to be determined may be from any process that produces a gas containing one or more polyhalogenated compounds. A gas may for example be flue gas from a combustion process or an incineration process, such as waste or biomass incineration process, or an exhaust gas from a metal production process, an industrial thermal process for energy or material conversion or any other industrial process.

The at least one sampler will continuously accumulate one or more polyhalogenated compounds from the gas that is brought into contact with sampler material during the sampling period as these substances will become absorbed and adsorbed by the sampler material. A time weighted average (TWA) concentration of the one or more polyhalogenated compounds may consequently be calculated over a sampling period using relatively simple calculations.

Furthermore, a method according to the present invention may be used to determine the concentration of one or more polyhalogenated compounds in a gas in any process producing a gas containing one or more polyhalogenated compounds as soon as the process starts to produce such a gas, i.e. during a start-up phase, and after operational disturbances. The at least one sampler used in a method according to the present invention may be arranged to sample gas continuously or for a long time, for example during the initial start-up phase of a process producing a gas containing one or more polyhalogenated compounds and throughout useful lifetime of the sampler material, i.e. up to five and a half years after the initial start-up phase. At least one sampler may namely be put in place before a process starts to produce a gas containing one or more polyhalogenated compounds and can remain in place during operational disturbances. Alternatively, a sampling period can be greater than one month, up to six months, up to one year, up to several years, or of any suitable duration, preferably in the order of one month to one year.

The step of calculating a concentration of one or more polyhalogenated compounds absorbed and adsorbed by the material from the gas to which the material was exposed may be carried out using the following equation:
concentration of one or more polyhalogenated compounds removed by the material from the gas [ng l-TEQ/Nm³]=(total weight of the material [g] *concentration of one or more polyhalogenated compounds in the material [ng l-TEQ/g])/total gas volume to which the material was exposed during the sampling period [Nm³]

If the at least one sampler is always in operation when a gas containing one or more polyhalogenated compounds is being produced, including during the start-up phase and during partial load operation, the total amount of polyhalogenated compounds absorbed and adsorbed by the at least one sampler correlates to the concentration of polyhalogenated compounds in the gas to which the material of the at least one sampler was exposed during the entire operating period. By plotting the polyhalogenated compound concentration in the sampler material versus time, indications about any change in polyhalogenated compound concentration during different periods, as well as indications of a decreasing absorption rate can be determined.

The material's polyhalogenated compound-absorption and adsorption efficiency may be measured using any suitable method, such as a conventional isokinetic gas sampling method. The material's polyhalogenated compound-absorption and adsorption efficiency can also be measured by placing material in the gas upstream and downstream of the material and determining the amount of polyhalogenated compounds absorbed and adsorbed by the material, for example by chemical analysis after its exposure to gas containing one or more polyhalogenated compounds.

Experiments have shown that the amount of polyhalogenated compounds absorbed and adsorbed by the material corresponds to the concentration of polyhalogenated compounds in the gas. If the polyhalogenated compound concentration in a material at a gas inlet is a hundred times higher than that at a gas outlet, then the mean outlet gas concentration can be estimated to be one hundredth of the inlet gas concentration, implying a mean removal efficiency of 99%.

The claimed method avoids the substantial cost and effort of using continuous isokinetic sampling methods, which are typically used today. The method utilizes a material with a high selectivity and high absorption and adsorption rate for polyhalogenated compounds and which is chemically resistant to many types of gas.

According to an embodiment of the invention the at least one sampler is a passive sampler. The term “passive sampling” means any sampling technique based on free flow of analyte molecules from the sampled medium to a receiving phase in a sampling device, as a result of a difference between the chemical potentials of the analyte in the two media. The net flow of analyte molecules from one medium to the other continues until equilibrium is established in the system, or until the sampling period is stopped. Sampling proceeds without the need for any energy source other than this chemical potential difference. A passive sampler for use in a method according to the present invention is therefore simple and inexpensive to manufacture and use, and very robust and reliable compared to existing isokinetic sampling devices.

According to an embodiment of the invention the polymer matrix is a polyolefin matrix, such as a polypropylene (PP) matrix, and/or the filler comprises carbon, preferably activated carbon, soot or ground hearth-furnace coke. The filler may be in particle, granulate or powder form.

According to an embodiment of the invention the filler that is suitable for adsorbing one or more polyhalogenated compounds which is uniformly distributed through the polymer matrix.

According to an embodiment of the invention the material is formed into one or more solid shapes, such as one or more rods, fibres, chips, granules, spheres, sheets or a moulded part produced by injection moulding, and arranged in the gas whose concentration of one more polyhalogenated compounds is to be determined. The material may be suspended in a gas flow using one or more wires, held in place by one or more retaining elements, or placed inside a container. The material of a sampler may be of any shape or size. The material can constitute the entire sampler. Tower packings, mist eliminators, test rods and dry, or semi-wet fixed bed packings of the material or may be used as samplers in a method according to the present invention.

According to an embodiment of the invention the method comprises the step of exposing all, or a substantial amount, of the gas whose concentration of one or more polyhalogenated compounds is to be determined to the material of the at least one sampler.

According to an embodiment of the invention the method comprises the step of passing the gas containing one or more polyhalogenated compounds through a plurality of samplers, and then mixing the plurality of samplers together for analysis as a single sample. Such an embodiment provides an advantage over conventional isokinetical sampling methods in that it is possible to measure a mean value of the concentration at many positions simultaneously, which will provide a more representative value for all of the gas. In the case of conventional isokinetic sampling, it is possible to traverse a probe over a gas flow cross section, typically in one or two traversing lines, when using a short sampling period, but the gas is still only sampled at one position at a time.

According to an embodiment of the invention the one or more polyhalogenated compounds is/are at least one of the following: polychlorinated dibenzo-p-dioxins, dibenzofurans (PCDD/Fs), polychlorinated biphenyls (PCBs), where the latter normally contributes very little to the total toxic equivalent (TEQ) value.

According to an embodiment of the invention the method comprises the step of extracting a sample of the material from a sampler and analysing its surface to determine whether any surface scaling, such as a CaF₂ or CaSO₄ deposit, is present. This can be done by analysing the material surface optically or using a Scanning Electron Microscope (SEM) or Energy Dispersive X-ray Spectroscopy (EDS) The presence of surface scaling will adversely affect the material's polyhalogenated compound-absorption and adsorption efficiency and such material should be replaced or the material's polyhalogenated compound-absorption and adsorption efficiency should be re-calculated or re-measured. The present invention also concerns a computer program product that comprises a computer program containing computer program code means arranged to cause a computer or a processor to execute all of the calculation steps of a method according to any of the embodiments of the present invention, stored on a computer-readable medium or a carrier wave.

The present invention further concerns the new use of a material comprising a polymer matrix and a filler that is suitable for adsorbing one or more polyhalogenated compounds which is distributed through the polymer matrix for determining a concentration of one or more polyhalogenated compounds in a gas. This known material has previously only been used in gas-cleaning systems, such as in scrubber systems to remove polyhalogenated compounds from gases in industrial exhaust steams. In the present invention, the material is used in a sampling device as a detector element in cases where the claimed method is used to detect the presence of one or more polyhalogenated compounds in a gas, or as sampling element in cases where the claimed method is used to determine the concentration of one or more polyhalogenated compounds in a gas.

The method according to the present invention will not only allow an accurate determination or estimate of the concentration of one or more polyhalogenated compounds in a gas to be made; but will also simultaneously clean the gas by removing one or more polyhalogenated compounds from the gas.

### BRIEF DESCRIPTION OF THE DRAWINGS

The present invention will hereinafter be further explained by means of non-limiting examples with reference to the appended figures where;

FIG. 1 shows how polyhalogenated compounds accumulate with time in a sampler material used in a method according to the present invention,

FIG. 2 shows the PCDD/F concentration in samples extracted after different exposure times, up to 2000 days (i.e. up to 5.5 years),

FIG. 3 schematically shows a plurality of samplers being used in a method according to an embodiment of the invention,

FIG. 4 shows PCDD/F concentrations according to Example 1 versus PCDD/F concentrations measured by extractive long-term isokinetic sampling, and

FIG. 5 shows PCDD/F concentrations according to Example 2 versus PCDD/F concentrations measured by extractive short-term isokinetic sampling.

It should be noted that the drawings have not necessarily been drawn to scale and that the dimensions of certain features may have been exaggerated for the sake of clarity.

### DETAILED DESCRIPTION OF EMBODIMENTS

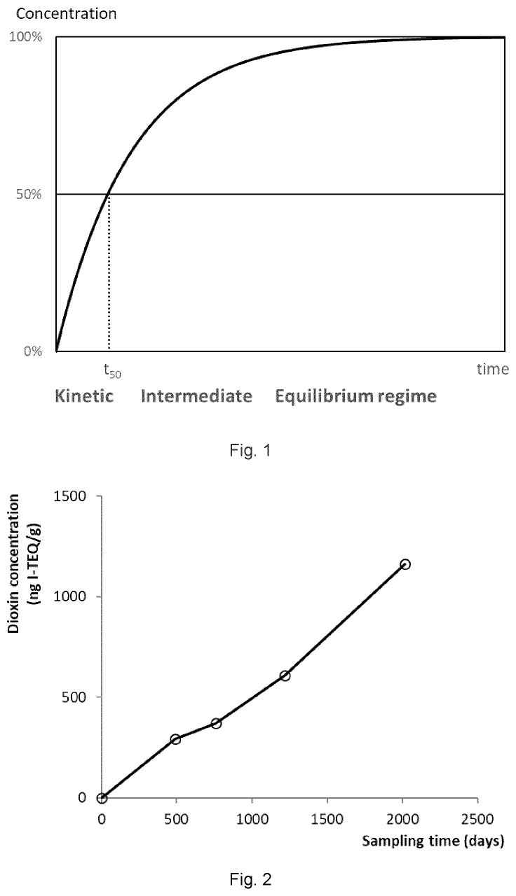

FIG. 1 shows how the concentration of polyhalogenated compounds in a sampler material changes with time. The accumulation of one or more polyhalogenated compounds in the sampler is limited by kinetics in a first regime. After a sufficient amount of time, the concentration of the one or more polyhalogenated compounds reaches equilibrium.

Since the material (as described in U.S. Pat. No. 7,022,162 B2) has been used in more than 120 incineration lines worldwide in order to remove PCDD/Fs from gas flows, a large number of time-concentrations have been recorded.

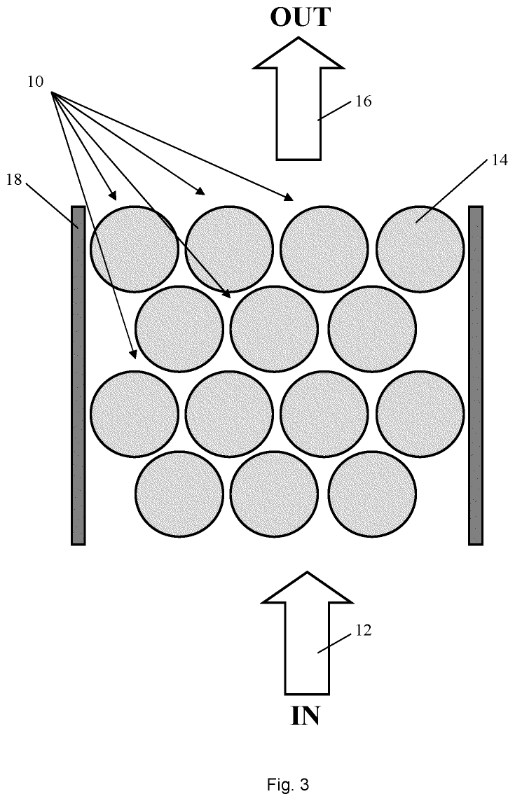

FIG. 2 shows the PCDD/F concentration in samples extracted after different exposure times up to 2000 days (i.e. up to 5.5 years) and shows that the absorption rate was fairly constant over a period of 2000 days. There is a linear dependence of concentration with sampling time and FIG. 2 shows that a sampler can operate in the kinetic regime for several years. The at least one sampler used in a method according to the present invention is preferably arranged so that it/they operate(s) in the kinetic regime.

FIG. 3 shows a plurality of samplers 10 being used in a method for determining a concentration of one or more polyhalogenated compounds in a gas according to an embodiment according to the present invention. The samplers 10, which are preferably passive samplers operating in the kinetic regime, are arranged in a gas channel 18 to sample a gas 12 containing one or more polyhalogenated compounds. The samplers 10 comprise a polymer matrix that is suitable for absorbing one or more polyhalogenated compounds and a filler that is suitable for adsorbing one or more polyhalogenated compounds which is distributed through the polymer matrix. The sampler material 14 is exposed to a gas 12 containing one or more polyhalogenated compounds which flows through the gas channel, whereby the gas 12 is the gas whose concentration of one or more polyhalogenated compounds is to be determined.

The polymer matrix of the material 14 may comprise a polyolefin matrix, such as a polypropylene (PP) matrix, and/or the filler of the material 14 may comprise carbon, preferably activated carbon, soot or ground hearth-furnace coke or any combination thereof. The filler content in the polymer matrix may be in the range of 0.1 to 30 weight-%.

The filler may be in particle, granulate or powder form or any combination thereof. The material 14 may be formed in any suitable shape and size retained or contained in one or more containers of any suitable shape and size. In the illustrated embodiment the samplers are in the form of spherical filler bodies located in a gas channel 18 in order to maximise the surface area of material 14 exposed to the gas flow and to permit a flow-through of gas 12. Conventional scrubber tower packings have a shape that is suitable for such an embodiment. Injection-molding may for example be used for producing the material 14. A dual screw-type extruder is for example is suitable for mixing the filler with the polymer matrix. If a plurality of samplers 10 are used, not all of the sampler material 14 which has been exposed to a gas 12 needs to be sent for chemical analysis in order to determine an amount of one or more polyhalogenated compounds adsorbed or absorbed by the sampler material 14. It is sufficient to send at least one part of the material 14 of at least one sampler 10, or the entire material from one or many samplers 10, such as from all samplers 10, for analysis. According to an embodiment of the invention, just one sampler 10 is used in the claimed method.

By determining the amount of one or more gaseous polyhalogenated compounds absorbed and adsorbed by the material 14, and measuring or calculating the material's polyhalogenated compound-absorption and adsorption efficiency, the concentration of one or more gaseous polyhalogenated compounds in the gas 12 to which the material 14 has been exposed, the concentration of one or more polyhalogenated compounds in the gas to which the material was exposed may be calculated.

The concentration of polyhalogenated compounds in gas upstream and downstream of the material 14 may be calculated as outlined below:

Cᵢₙ=C_{removed}/η

Cₒᵤₜ=(1−η)*Cᵢₙ

wherein:
η=the material's polyhalogenated compound removal efficiency;
Cᵢₙ=concentration in gas upstream of the material;
C_{removed}=polyhalogenated compound concentration removed from the gas; and
Cₒᵤₜ=concentration in gas downstream of the material.

The method according to the present invention may comprise the step of sampling all of the gas 12 whose concentration of one or more polyhalogenated compounds is to be determined by exposing the material 14 of at least one sampler 10 to all of the gas 12.

Alternatively, the method may comprise the step of exposing the material 14 of at least one sampler 10 to only a fraction of a gas 12, whose concentration of one or more polyhalogenated compounds is to be determined.

According to an embodiment of the invention the method comprises the step of passing a gas 12 containing one or more polyhalogenated compounds through a plurality of samplers 10, and then mixing the outcoming gas 16 that has passed though the plurality of samplers 10 together for analysis as a single sample. The plurality of samplers 10 may be arranged at different positions across a gas flow cross-section, such as at different positions along the diameter of a flue gas channel.

According to an embodiment of the invention the method comprises the step of extracting a sample of the material 14 from the at least one sampler and analysing its surface to determine whether any surface scaling is present. If a sampler is partly or intermittently exposed to an aqueous solution supersaturated with an inorganic compound, then a scaling of the inorganic component may be formed on the surface on the sampler material 14, which may adversely affect the absorption and adsorption rate.

In order to monitor and optimize the lifetime and performance of the material 14 and ensure that accurate determinations or estimations of the concentration of polyhalogenated compounds in a gas are being made, samples of material 14 may be extracted from a sampler 10 and the polyhalogenated compound concentration, surface scaling and mechanical properties of the sample of material 14 may be checked. It has been found that the mean service life of the material 14 is six years.

The calculations required to determine a concentration of one or more polyhalogenated compounds in a gas may be executed by a computer or a processor using a computer program product according to the present invention.

### Example 1

A random tower packing produced from sampler material 14 was secured by a polytetrafluoroethylene (PTFE) wire and inserted into the gas duct downstream of a wet scrubber of a flue gas treatment system where it was exposed to a gas flow. After a sampling period of one month, the tower packing was extracted from its exposed position and analyzed for PCDD/Fs. Thereby, the PCDD/F concentration in the flue gas could be calculated.

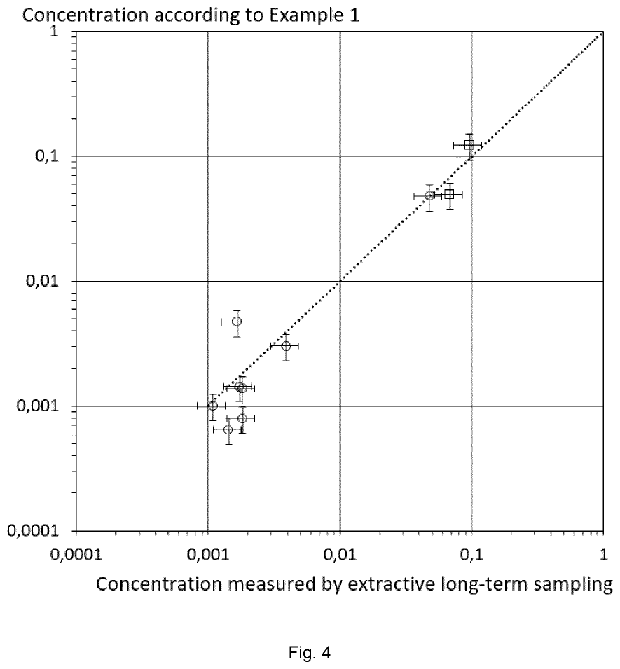

FIG. 4 shows PCDD/F concentrations in ng I-TEQ/Nm³ d.g. (act. O₂) determined for two different plants, marked with squares and circles, respectively. The graph shows good agreement between in-situ sampled PCDD/F concentrations as measured according to the inventive Example 1 on the y-axis, versus PCDD/F concentrations as measured using extractive long-term isokinetic sampling for the same two plants on the x-axis. The error bars show the estimated measurement uncertainties. In some cases, the long-term sampler had not been operating during the entire sampling period of the sampler according to Example 1, due to maintenance or automated idle mode.

### Example 2

Random tower packings produced from sampler material 14 were installed in one or more wet scrubber stages of a flue gas treatment system in such a manner, that a significant fraction, such as 20-99%, of PCDD/Fs was separated from the entire flue gas. After a certain time, for example one year of operation, a number of packing elements were extracted from each stage and analyzed for PCDD/Fs. By applying a mass balance to the total amount of sampler material 14 and the total volume of gas that had passed through the material during the sampling period, the concentration of polyhalogenated compounds absorbed and adsorbed by the material was calculated. The PCDD/F removal efficiency was measured or calculated and the input and output concentrations were calculated. For comparison, the material's polyhalogenated compound-absorption and adsorption efficiency can also be measured using extractive short-term isokinetic gas sampling methods. The material's polyhalogenated compound-absorption and adsorption efficiency can furthermore also be measured by placing sampler material 14 in the inlet and outlet gas, i.e. the gas 12 upstream and the gas 16 downstream of the samplers 10.

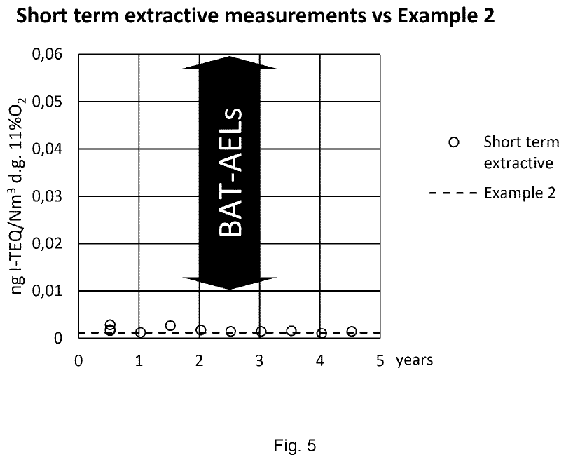

FIG. 5 shows that the PCDD/F concentrations sampled for 5 years according to Example 2, marked by a dashed line (---), and the PCDD/F concentrations measured by comparative extractive short-term isokinetic sampling, marked (∘), are in good agreement. The concentrations are given in ng I-TEQ/Nm³ d.g. (11% O₂).

In Example 1, the material of the single random tower packings was exposed to only a part of the gas whose concentration of one or more polyhalogenated compounds was to be determined.

In Example 2, the material of the plurality of random tower packings was exposed to a substantial amount of the gas whose concentration of one or more polyhalogenated compounds was to be determined.

There are very large economic incitements for large plants to run near 100% of their capacity for most of the time, typically 8000 hours per year. The variations of the gas flow over time are typically relatively small, typically in the order of ±10-20% for the majority of the time. Moreover, the temperature of the gas flow is usually quite constant (typically plus or minus a few degrees Centigrade). These circumstances are ideal for determining a concentration of one or more polyhalogenated compounds in a gas flow using a method according to the present invention accurately, since the absorption and adsorption rate of one or more polyhalogenated compounds into/onto the material may be temperature and gas velocity dependent.

Further modifications of the invention within the scope of the claims would be apparent to a skilled person. For example, any feature disclosed with reference to one embodiment of the invention may be combined with one or more features of another embodiment of the invention unless such a combination is explicitly excluded.

1. A method for determining a concentration of one or more polyhalogenated compounds in a gas, wherein the method comprises the steps of
exposing at least one sampler containing or constituted by a material comprising a polymer matrix that is suitable for absorbing one or more polyhalogenated compounds, and a filler that is suitable for adsorbing one or more polyhalogenated compounds which is distributed through said polymer matrix, to gas containing one or more polyhalogenated compounds during a sampling period, whereby said gas constitutes at least part of said gas whose concentration of one or more polyhalogenated compounds is to be determined,
determining an amount of one or more polyhalogenated compounds adsorbed or absorbed by said material, and
calculating a concentration of one or more polyhalogenated compounds in said gas to which said material was exposed, either upstream or downstream of said at least one sampler.

2. The method according to claim 1, wherein it comprises the steps of measuring or calculating said material's-polyhalogenated compound absorption and adsorption efficiency, and calculating a concentration of one or more polyhalogenated compound in the gas whose concentration of one or more polyhalogenated compounds is to be determined.

3. The method according to claim 1, wherein said at least one sampler is a passive sampler.

4. The method according to claim 1, wherein said polymer matrix is a polyolefin matrix, such as a polypropylene matrix, and/or said filler comprises carbon, preferably activated carbon, soot or ground hearth-furnace coke.

5. The method according to claim 1, wherein said filler is in particle, granulate or powder form.

6. The method according to claim 1, wherein comprises the step of sampling all, or a substantial amount of the gas whose concentration of one or more polyhalogenated compounds is to be determined by exposing said material of the at least one sampler to all of the gas whose concentration of one or more polyhalogenated compounds is to be determined.

7. The method according to claim 1, wherein it comprises the step of passing said gas containing one or more polyhalogenated compounds through a plurality of samplers, and then mixing said plurality of samplers together for analysis as a single sample.

8. The method according to claim 1, wherein said one or more polyhalogenated compounds is/are at least one of the following: polychlorinated dibenzo-p-dioxins, dibenzofurans (PCDD/Fs), polychlorinated biphenyls (PCBs).

9. The method according to claim 1, wherein comprises the step of extracting a sample of said material and analysing its surface to determine whether surface scaling is present.

10. A non-transitory computer readable medium storing a computer program containing computer program code that causes a computer or a processor to execute all of said calculation steps of a method according to claim 1.

11. A material comprising a polymer matrix that is suitable for absorbing one or more polyhalogenated compounds and a filler that is suitable for adsorbing one or more polyhalogenated compounds which is distributed through said polymer matrix for determining a concentration of one or more polyhalogenated compounds in a gas to which said material is exposed.
